(19) United States
(12) Patent Application Publication
(10) Pub. No.: US 2002/0187482 A1
(43) Pub. Date: Dec. 12, 2002

(54) Methods and means of RNA analysis

(76) Inventors: Zicai Liang, Sundbyberg (SE); Hong-Yan Zhang, Sundbyberg (SE); Claes Wahlestedt, Stockholm (SE)

Correspondence Address: MARK S. ELLINGER, PH.D., Fish & Richardson P.C., P.A., Suite 3300, 60 South Sixth Street, Minneapolis, MN, 55402, US

(21) Appl. No.: 09/961,700

(22) Filed: Sep. 24, 2001

Related U.S. Application Data
(60) Provisional application No. 60/235,029, filed on Sep. 25, 2000.

(51) Int. Cl. C12Q 1/68; C12P 19/34

(52) U.S. Cl. 435/6; 435/91.2

(57) ABSTRACT

This invention relates to methods for identifying regions of RNA molecules that are available for interaction with small molecules, particularly regions that can hybridize with oligonucleotides having complementary sequences. Identifying such regions is useful in the design of probes, anti-sense oligonucleotides and small molecule drugs.

### RELATED APPLICATION

This application claims the benefit of U.S. provisional application serial No. 60/235,029, filed Sep. 25, 2000.

### TECHNICAL FIELD

This invention relates to methods for the identification of regions within RNA molecules that are available for interaction with small molecules, particularly regions that can hybridize with oligonucleotides having complementary sequences. The identification of such regions is useful in the design of probes, anti-sense oligonucleotides and small molecule drugs.

### BACKGROUND

Messenger RNA (mRNA) is information-carrying intermediate in protein synthesis that is transcribed by RNA polymerase from a DNA template and subsequently translated by ribosomes to generate protein molecules. Anti-sense oligonucleotides are commonly used to disrupt mRNA function. These are short nucleic acid molecules that have a sequence complementary to that of an mRNA molecule. By pairing with their cognate mRNA sequences in vivo, antisense oligonucleotides (DNA and modifications, such as LNA and PNA) can specifically modulate gene expression. Mechanisms for this modulation may include aberration of splicing and/or translation, or destabilization of the target mRNA (1, 2).

Antisense technology is becoming one of the most useful tools in functional genomics, at a time when a large number of gene sequences has been generated in the genome projects. The method can also be readily configured to work with unknown genes in any species. Other potential advantages of the technology include the simultaneous targeting of multiple genes, and the identification of drug candidates directly from the gene sequences.

mRNA molecules are normally folded into complex secondary and tertiary structures upon synthesis, leaving only small patches of sequences which are relatively accessible for binding with foreign sequences such as antisense oligonucleotides. Rules governing such accessibility differences have not been established, and experimental tools have been the major approaches that can be used to predict the accessible regions of a given mRNA.

The lack of efficient and cost-effective methods of selecting antisense sequences that gain access to the RNA target has hindered the application of antisense technology. Most active antisense oligonucleotides have been chosen empirically using in vitro or in vivo assays and only a small proportion of the tested antisense oligonucleotides (normally 2-10%) exhibit sequence-specific activities. Several experimental procedures have been developed for the prediction of regions of mRNA sequence that are available for antisense oligonucleotide binding (3, 4, 5, 6, 7). The practical complexities of these methods and/or poor availability of the tools have prevented the widespread use of the methods. Computational approaches have also been used for such predictions (8, 9, 10), but application of these prediction models beyond the training set of genes is still questionable.

Many novel human genes have been uncovered and more will be identified in the very near future. Similarly, large numbers of gene sequences from other species are also becoming available continuously. None of the existing methods can offer the throughput to resolve the mRNA accessibility of more than a fraction of these genes.

### SUMMARY

The present invention relates to a simple bench-top method, known as “mRNA Accessible Site Tagging” (MAST), which provides high throughput mapping of mRNA accessibility using standard molecular biology procedures. This method provides for the simultaneous study of the RNA accessibility of any number of RNA sequences.

Empirical testing and experimental assays are widely used for predicting effective antisense sequence. Existing experimental methods suffer from cumbersome procedures and low throughput. The MAST method described herein is simple and easy to perform in any laboratory equipped for standard molecular biology work. Thorough interrogation of a small number of mRNA (10 mRNAs for example) can be done within less than a week using this method. This throughput level should meet most laboratory needs in terms of anti sense sequence selection. The MAST procedure is designed so that experiments can be easily scaled up. No adjustment is needed in MAST procedure when performing analysis on multiple mRNA or mRNA or different length. It is theoretically possible to use this method to investigate tens to hundreds of mRNA in the same reaction tube, thus affording unrivalled parallel processing capacity in mRNA accessibility analysis.

### DESCRIPTION OF DRAWINGS

FIG. 1 shows a diagram of mRNA accessibility for antisense oligonucleotide binding.

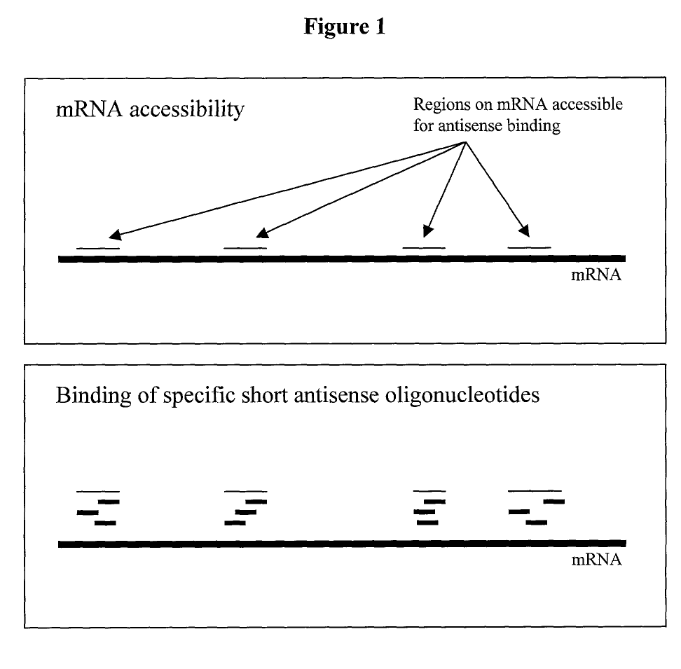

FIG. 2 shows an example of a random oligonucleotide library suitable for use in methods of the present invention. A short (8-30 nt) randomized single-stranded oligonucleotide sequence was nested in between two stretches of known sequences. The known sequences were designed to facilitate subsequent PCR amplification of the library while not interfering with the hybridization of the single-stranded region. 15-mer and 18-mer libraries have been tested. Amplification strategies are shown in A and B and cloning and sequencing strategies are shown in C.

FIG. 3 shows a first scheme of di-tag synthesis. A library is amplified as shown with two different 3′ primers (A) and then cleaved with 5′ tagging enzyme (BamHI as demonstrated). The cleaved fragments are then dimerized by T4 DNA ligase. Di-tags are amplified with two primers that are nested to the two 3′ primers. The generation of di-tags can be one strategy for more efficient concatemerization.

FIG. 4 shows a second scheme of di-tag synthesis. A library contains two sub-libraries, each have a single-stranded randomized sequence (8-30 nt) and long (15-30 nt) 3′ double-stranded sequence (priming sites). The two sub-libraries differ in their 3′ double-stranded regions (A). After MAST selection, the selected molecules are rendered double-stranded by enzymatic fill-in and then dimerized by blunt-end ligation (B). Di-tags having two different priming sites can then be PCR amplified (C).

FIG. 5 is a schematic diagram of the MAST method. FIG. 5A shows an oligonucleotide library with an 18 nt stretch of fully randomized sequence nested between two PCR priming sites. Both priming sites are then blocked by annealing to their complementary blocking oligonucleotides, leaving only the random portion single-stranded. Priming site B is designed to be truncated to afford more flexibility to the single-stranded region. FIG. 5B shows a biotin labeled mRNA synthesized by in vitro transcription and bound to streptavidin coated paramagnetic beads. The oligonucleotide library is allowed to hybridize with the immobilized mRNA under controlled temperature and salt concentration. After unbound and non-specifically bound oligonucleotides are removed by washing at proper stringency, oligonucleotides that specifically bound to the mRNA are eluted by boiling in H₂O and referred to as AST (Accessible Site Tags). FIG. 5C shows the AST annealed to a site B specific primer and rendered double-stranded by enzymatic fill-in. The truncated priming site B is rebuilt into a full priming site at this step. The AST is PCR amplified and cloned into vectors for normal or high throughput sequencing.

FIG. 6 shows MAST mapping of the first 122 nt of rabbit β-globin mRNA. Two regions with significant accessibility were identified by multiple ASTs. ASTs share identities within the accessible regions, but have diverse sequence characteristics outside the accessible regions. This helps to precisely define the location of accessible region. Italicized, double underlined letters show wobbling locations where one additional nt was observed in the AST.

FIG. 7 shows MAST mapping of β-galactosidase mRNA. A 1 kb fragment of β-galactosidase mRNA was used in this experiment and clusters of AST suggest that four regions (underlined) in this mRNA fragment appear to be accessible for antisense binding. Italicized, double underlined letter shows a wobbling nt in the duplex.

FIG. 8 shows MAST mapping of mRNA encoding a novel G protein-coupled receptor CGR95. Five regions in CGR95 were indicated by multiple ASTs to be open of antisense binding. The effective sequence overlaps largely with a single AST tag where as none of the nine sequences with negative results overlap with any of the AST tags.

FIG. 9 shows in vitro antisense activity assays in HEK 293 cells.

FIG. 10 shows in vivo effects of antisense oligonucleotides (50.0 mg, twice a day, i.c.v.) targeted against a brain orphan G-protein coupled receptor on locomotor behaviour in rats.

### DETAILED DESCRIPTION

A first aspect of the present invention provides a method for identifying an accessible region in a test RNA molecule, comprising: bringing into contact a test RNA molecule and a population of oligonucleotide molecules under conditions in which the test RNA molecule retains its native structure, each oligonucleotide molecule in the population comprising a portion consisting of random nucleotides, whereby said portion of each oligonucleotide is able to bind to a complementary accessible region of an RNA molecule if present; selecting an oligonucleotide molecule which binds to the test RNA molecule at an accessible region of the test RNA molecule; determining the sequence of said portion of the selected oligonucleotide molecule; and identifying the sequence of the accessible region of the test RNA molecule to which said selected oligonucleotide molecule binds. RNA suitable for use in methods of the present invention includes mature mRNA, pre-mRNA and any other RNA.

The mRNA molecule or the population of oligonucleotides may be immobilized. Suitable methods of immobilizing are well known in the art and may include covalent or non-covalent attachment to a microplate well, microfuge tube, magnetic bead or other glass or plastic bead or surface.

Oligonucleotide which binds to the test mRNA molecule may be selected by separating the immobilized test mRNA and oligonucleotide bound to it from unbound oligonucleotide. This may be carried out using any known method. Conveniently, the mRNA molecule may be attached to a magnetic bead and separation achieved using a magnet. Attachment of macromolecules to magnetic beads is well known in the art and may be achieved, for example, using a biotinylated mRNA molecule and a streptavidin-coated bead according to standard protocols. Alternatively, test mRNA that binds to the oligonucleotide molecule may be selected by separating the immobilized oligonucleotide and test mRNA bound to it from unbound oligonucleotide and mRNA. Conveniently, mRNA may be labeled, for example, using fluorescent dye, radioactive label or affinity labels such as biotin or antigen. Oligonucleotides may conveniently be attached to beads. Beads labeled through oligonucleotide/mRNA binding with label may be separated from unlabelled beads using flow cytometry selection or affinity selection. Attachment of macromolecules to beads is well known in the art and may be achieved, for example, using a biotinylated oligonucleotide molecule and a streptavidin-coated bead according to standard protocols. The sequence of immobilized oligonucleotide may then be determined as described herein.

A library of oligonucleotides as described herein may be used to identify accessible regions on mRNA molecules of different sequences. This offers a significant advantage over known methods, in which oligonucleotides specific for a particular mRNA must be synthesized.

The portion of random nucleotides may consist of between 13 and 18 random bases, more preferably 15 to 18 random bases, for example 15, 16, 17 or 18 random bases. The random portion should be long enough to hybridize to cognate mRNA under physiological conditions, allowing the mRNA to maintain its physiological conformation. Terminal nucleotides at both the 3′ and 5′ ends of the random portion do not always take part in hybridization. This may reduce the effective length of the random portion of a library having 15 to 18 random bases to the range of 13 to 15 random bases.

Preferably the population of oligonucleotide molecules consists of a library of such molecules in which all the possible sequences of the random portion are represented (i.e. for any possible sequence of the random portion, there is at least one molecule present in the library which has a random portion consisting of that particular nucleic acid sequence).

Methods of the present invention may be used in high throughput analysis. Binding oligonucleotides may be selected from an oligonucleotide library by multiple mRNA molecules of different sequence simultaneously in the same reaction medium. Accessible regions of each different mRNA molecule may then be individually identified by comparing each mRNA sequence with all the selected oligonucleotides. The reaction medium may contain a plurality of mRNA molecules having different sequences, for example 2 to 500 mRNA molecules, preferably 10 to 50, for example 5, 10, or 20 mRNA molecules. Using an oligonucleotide library with 15 to 18 random bases (i.e. an effective random portion of 13 to 15 bases) with a threshold of 70% similarity as for sequence identification, about 50 to 100 kb of mRNA may be accommodated in each batch. This equates to a range of about 30 to 60 average mRNA molecules. Increasing the identification threshold will increase the number of different mRNA molecules that can be accommodated (e.g. up to 1000-2000). However, the rejection rate (i.e. the disposal of oligonucleotides which are only slightly different to the mRNA sequence) would also increase, with a consequent increase in sequencing costs.

The oligonucleotide library is contacted with the RNA molecule in the reaction medium under conditions that allow the binding of oligonucleotide without disrupting the secondary and tertiary structure of the mRNA molecule. Suitable conditions include the presence in the medium of pH buffering agents (such as phosphate salts) and non-buffering salts or organic compounds which modulate the strand annealing properties of the nucleic acids. Detergents such as SDS and Tween-20 and carrier molecules such as complex DNA, tRNA and poly(da) may also be included in different proportions to minimize the non-specific interaction of the probes with the surface of the target nucleic acid molecule. Suitable low stringency conditions include hybridisation and washing at 37° C. to 40° C. in 1× to 5×SSC and 0.1% SDS, for example 40° C. in 2×SSC, 0.1% SDS.

Non-random nucleic acid sequence of the oligonucleotide molecules may be blocked during hybridisation by annealing to a blocking oligonucleotide having a complementary nucleic acid sequence using known methods. A molecule comprising double-stranded known sequence and single-stranded randomized sequence is thus formed. This molecule is then contacted with the mRNA molecule so that the single-stranded, randomized binding region is available to bind to the mRNA. The blocking oligonucleotide may prevent non-random sequence annealing to the mRNA and impeding hybridisation of the randomized sequence.

The sequence of the random portion of a selected oligonucleotide molecule may be determined by any method known in the art. Known methods include Sanger dideoxynucleotide termination, Maxam-Gilbert enzyme degradation, pyrosequencing, sequencing by hybridization, and gel capillary mass spectrometry.

The sequence of random nucleotides that binds to the RNA molecule will correspond to the sequence of the accessible region of the RNA molecule. The sequence of random nucleotides may be complementary to the RNA sequence or show 60% to 99% sequence identity to such a complementary sequence, for example 60%, 70%, 80%, 90%, 95% or 99% sequence identity. Exactly complementary sequences are sequences that show 100% complementarity to each other and will therefore anneal without any mismatch. Sequences may exhibit lesser degrees of complementarity. For example, 60% to 99% sequence identity to a complementary sequence corresponds to 60% to 99% complementarity. Under low stringency hybridisation conditions, exact complementarity (i.e. 100%) is not required in order for the randomized sequence to bind to the mRNA.

An accessible region of the test mRNA may be identified by comparing the sequences of the random portion of selected oligonucleotides that are found to bind to the mRNA with the known mRNA sequences. The comparison may be done using conventional algorithms as described herein. The accessible region will show complementarity with the random oligonucleotide sequence (i.e. the region will show sequence identity as disclosed herein with a sequence complementary to the random oligonucleotide sequence). An accessible region may show 60% to 99% sequence identity to such a complementary sequence, for example 60%, 70%, 80%, 90%, 95% or 99% sequence identity.

Sequence identity, homology and/or complementarity may be determined by computer using an appropriate algorithm or program. A preferred algorithm may be GAP, which uses the alignment method of Needleman and Wunsch (1970) J. Mol. Biol. 48: 443-453 and is included in the Program Manual of the Wisconsin Package, Version 8, September 1994 (Genetics Computer Group, 575 Science Drive, Madison, Wis., USA). In the absence of instructions to the contrary, the skilled person would understand to use the default parameters with the aim of maximizing alignment, with a gap creation penalty =12 and gap extension penalty =4.

Similarity or homology (the terms are used interchangeably) or identity may be as defined and determined by the TBLASTN program, of Altschul et al. (1990) J. Mol. Biol. 215: 403-10, or BestFit, which is part of the Wisconsin Package, Version 8, September 1994 (Genetics Computer Group, 575 Science Drive, Madison, Wis., USA, Wisconsin 53711). Preferably, sequence comparisons are made using FASTA and FASTP. See, Pearson & Lipman (1988) Methods in Enzymology 183: 63-98. Parameters are preferably set, using the default matrix, as follows: Gapopen (penalty for the first residue in a gap): −12 for proteins/−16 for DNA; Gapext (penalty for additional residues in a gap): −2 for proteins/−4 for DNA; KTUP word length: 2 for proteins/6 for DNA.

Normally, only a small proportion of the mRNA sequence is accessible although this varies from mRNA to mRNA. It is preferred that sufficient binding oligonucleotides are sequenced to enable an accessible region to be independently pinpointed by oligonucleotide sequences at least six times. This normally represents a total number of 20-40 oligonucleotides per mRNA molecule that need to be sequenced and related to the mRNA sequence.

Methods of the present invention may be used in high throughput analysis. Binding oligonucleotides may be selected from an oligonucleotide library by multiple mRNA molecules of different sequence simultaneously in the same reaction medium. Accessible regions of each different mRNA molecule may then be individually identified by comparing each mRNA sequence with all the selected oligonucleotides. The reaction medium may contain a plurality of mRNA molecules having different sequences, for example 2 to 500 mRNA molecules, preferably 10 to 50, for example 5, 10, or 20 mRNA molecules. Using an oligonucleotide library with 15 to 18 random bases (i.e. an effective random portion of 13 to 15 bases) with a threshold of 70% similarity as for sequence identification, about 50 to 100 kb of mRNA may be accommodated in each batch. This equates to a range of about 30 to 60 average mRNA molecules. Increasing the identification threshold will increase the number of different mRNA molecules that can be accommodated (e.g. up to 1000-2000). However, the rejection rate (i.e. the disposal of oligonucleotides which are only slightly different to the mRNA sequence) would also increase, with a consequent increase in sequencing costs.

A further aspect of the present invention provides a method for identifying an accessible region in a test mRNA molecule, comprising: bringing into contact a test mRNA molecule and a population of oligonucleotide molecules under conditions in which the test mRNA molecule retains its native structure, each oligonucleotide molecule in the population comprising a portion consisting of random nucleotides, whereby said portion of each oligonucleotide is able to bind to a complementary accessible region of an mRNA molecule if present; selecting an oligonucleotide molecule which binds to the test mRNA molecule at an accessible region of the test mRNA molecule; amplifying the said portion of the selected oligonucleotide molecule, determining the sequence of said amplified portion of the selected oligonucleotide molecule; and, identifying the sequence of the accessible region of the test mRNA molecule to which said selected oligonucleotide molecule binds.

The selected oligonucleotide may be amplified using a specific nucleic acid amplification reaction such as the polymerase chain reaction (PCR) (reviewed for instance in Innis et al. (eds.) PCR protocols: A Guide to Methods and Applications (1990) Academic Press, New York; Ehrlich (ed.), PCR technology (1989) Stockton Press, N.Y.; Mullis et al. (1987) Cold Spring Harbor Symp. Quant. Biol. 51:263; and Ehrlich et al. (1991) Science 252:1643-1650). PCR comprises steps of denaturation of template nucleic acid (if double-stranded), annealing of primer to target, and polymerisation. In the present methods, oligonucleotides that hybridise to the mRNA are used as template in the amplification reaction. Other specific nucleic acid amplification techniques include strand displacement activation, the QB replicase system, the repair chain reaction, the ligase chain reaction and ligation activated transcription. For convenience, and because it is generally preferred, the term PCR is used herein in contexts where other nucleic acid amplification techniques may be applied by those skilled in the art. Unless the context requires otherwise, reference to PCR should be taken to cover use of any suitable nucleic amplification reaction available in the art.

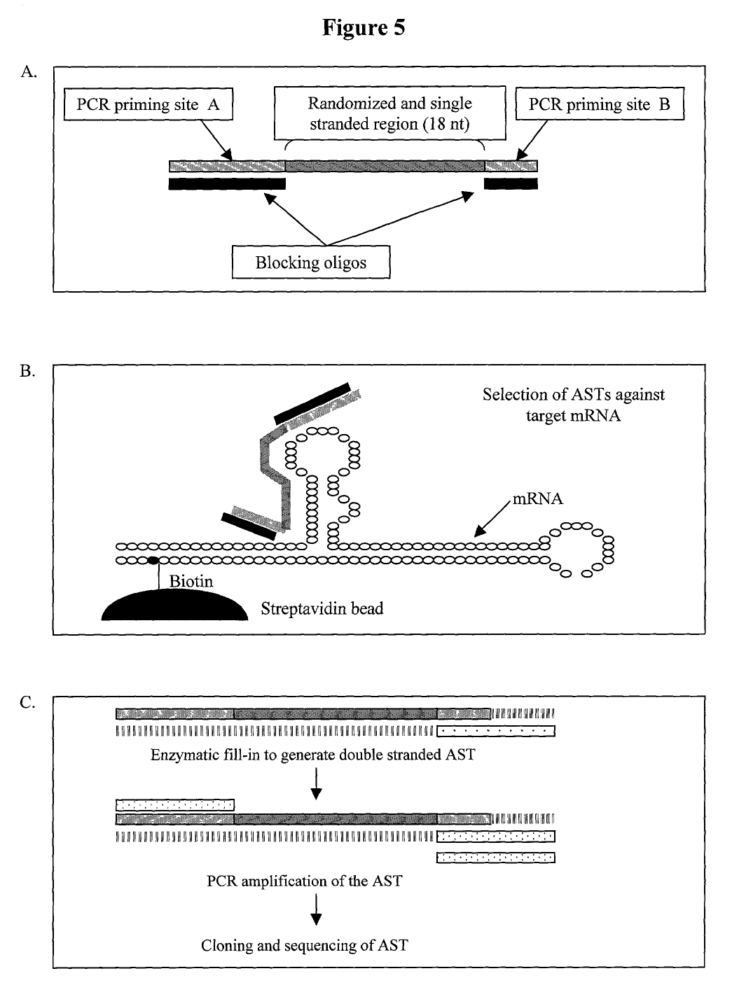

The oligonucleotide molecules in the library may further comprise a region of known, non-random, nucleic acid sequence (“clumping sequence”). This known sequence may be adjacent the random portion and may be used to amplify the randomized sequence prior to sequencing. Various arrangements of non-random sequence are possible according to the method used to amplify the randomized sequence. Non-random sequence may be located 5′ of the random sequence, 3′ of the random sequence or it may flank the random sequence (i.e. be located both 5′ and 3′ of the random sequence). 3′ non-random clumping sequence is preferably short, for example 4 to 10 nucleotides, preferably 5 to 7 nucleotides, to afford minimal steric hindrance and provide more flexibility to the random portion of the oligonucleotide. An oligonucleotide comprising a randomized sequence nested between a 5′ and a 3′ non-random sequence may be amplified using oligonucleotide amplification primers specific to the non-random sequence. Where the 3′ non-random sequence is short, a primer site may be re-built as shown in FIG. 5 by an enzymatic fill-in process using an oligonucleotide template. This may be achieved using techniques well known in the art, for example incubation with Taq polymerase as described herein. An amplification method such as Polymerase Chain Reaction (PCR) may be carried out on the single-stranded or the double-stranded template. Such methods are well known in the art.

An aspect of the present invention therefore provides a method involving: (a) obtaining a oligonucleotide using a method as described herein which binds to an mRNA; (b) providing a pair of nucleic acid molecule primers useful in (i.e. suitable for) PCR, at least one of said primers being a primer specific for a non-random sequence of the oligonucleotide; (c) contacting the oligonucleotide in the preparation with said primers under conditions for performance of PCR; and (d) performing PCR and determining the sequence of the amplified PCR product. Sequencing of a PCR product may involve precipitation with isopropanol, resuspension and sequencing using a TaqFS+ dye terminator sequencing kit. Extension products may be electrophoresed on an ABI 377 DNA sequencer and data analyzed using Sequence Navigator software.

Concatemerization of the selected oligonucleotides is one preferred way of achieving high throughout in this system. Another method that may facilitate high throughput is pyrosequencing. Amplification products may be conveniently analysed by concatemerising the amplification products. The concatemerised products may then be cloned and sequenced. When there is more than one mRNA molecule present in the reaction medium, concatemerisation allows the rapid sequencing of multiple oligonucleotides, each of which may bind to a different accessible region or a different mRNA. Accessible regions may be identified on each mRNA molecule by comparing the sequences of the selected oligonucleotides with the mRNA sequences.

A di-tag protocol may also be employed to improve the efficiency of concatemerisation. This may, for example, involve amplifying a selected oligonucleotide using a single 5′ amplification primer and two different 3′ primers. Following a first round of amplification, the amplification products (“first amplification products”) may be dimerised by cleaving within the 5′ primer sequence using a restriction enzyme and ligating the cleaved products together. The dimers thus produced, if they contain sequence corresponding to the two 3′ different primers at the ends, may then be amplified using primers nested to the two 3′ primers to generate further amplification products (“second amplification products”) for sequencing.

A short 5′ and 3′ non-random sequence may facilitate amplification of selected MAST tags but the double-stranded region that comprises the 3′ sequence and the 5′ blocking oligonucleotide might interfere with hybridization to the mRNA target. In order to eliminate this possibility, a di-tag approach similar to that described above may also be employed with a library of oligonucleotide molecules comprising a region of randomized sequence and a 3′ non-random known sequence. Such a library may comprise two sub-libraries, each having a different 3′ known region. After selection, the oligonucleotide molecules are rendered double-stranded by enzymatic fill-in and dimerised by blunt ended ligation. Those dimerised “di-tag” molecules having different primer sites at each end may then be amplified using conventional techniques.

Methods of the present invention provide for identification of accessible regions in an mRNA by sequence comparison with the random portions of oligonucleotides from the population that bind to the mRNA. As disclosed herein, the random portion may show complementarity, such as 60%, 70%, 80%, 90% or 95% to the sequence of the accessible region. The accessible regions thus identified are suitable targets for anti-sense oligonucleotides, which can be designed to be complementary (i.e. show 100% complementarity) to the accessible region.

Anti-sense oligonucleotides may be designed to hybridise to the complementary sequence of accessible regions of nucleic acid, pre-mRNA or mature mRNA as identified herein, interfering with the production of polypeptide encoded by a given DNA sequence (e.g. either native polypeptide or a mutant form thereof), so that its expression is reduce or prevented altogether. Anti-sense techniques may be used to target a coding sequence, a control sequence of a gene, e.g. in the 5′ flanking sequence, whereby the antisense oligonucleotides can interfere with control sequences. Anti-sense oligonucleotides may be DNA or RNA and may be of around 7-40 nucleotides, particularly around 10-18 nucleotides, in length. The construction of antisense sequences and their use is described in Peyman and Ulman (1990) Chemical Reviews 90:543-584, and Crooke (1992) Ann. Rev. Pharmacol. Toxicol. 32:329-376.

An anti-sense oligonucleotide may be DNA, RNA or PNA (protein nucleic acid) and may be modified to increase its resistance to endogenous cellular nucleases. Any nucleic acid molecule such as an oligonucleotide that is used in a biological context is subject to the degradative action of the cell nucleases, thus a variety of modifications have been evolved to protect oligonucleotides, the most commonly used of which is the introduction of phosphorothioate (PS) analogues (Stein and Cheng, 1993), which have sulphur in place of one of the non-bridging oxygen atoms bonded to phosphorous. This modification confers resistance to nucleases while maintaining the ability to elicit RNase H activity (Agrawal S. (1996) Trends in Biotechnology 14:376). Alternative stabilizing approaches may be tested to improve the nuclease resistance of a nucleic acid molecule. Other new classes of oligonucleotide backbone modification are currently being developed to avoid possible liver toxicity in humans with PS (reviewed in Agrawal S. and Iyer R. P. (1995) Curr. Opin. Biotechnology 6:12). In addition to backbone and sugar modifications, the heterocyclic bases may also be modified (Agrawal & Iyer, as above).

Various techniques for synthesizing oligonucleotides are well known in the art, including phosphorothioate, phosphotriester and phosphodiester synthesis methods. It is desirable that the antisense oligonucleotide is resistant to nuclease digestion and this can be achieved by known methods of inter-base modification.

Many known techniques and protocols for manipulation of nucleic acid, for example in preparation of nucleic acid constructs, mutagenesis, sequencing, introduction of DNA into cells and gene expression, and analysis of proteins, are described in detail in Ausubel et al. (eds.) Current Protocols in Molecular Biology: Second Edition (1992) John Wiley & Sons, and in Sambrook et al. Molecular Cloning: A Laboratory Manual: 2nd edition (1989) Cold Spring Harbor Laboratory Press. The disclosures of Sambrook et al. and Ausubel et al. are incorporated herein by reference.

A further aspect of the present invention provides a method of manufacturing an anti-sense oligonucleotide for the down-regulation of expression from an mRNA comprising: identifying an accessible region on an mRNA using a method described herein, and synthesising an oligonucleotide complementary to said accessible region. A further aspect of the present invention provides an anti-sense oligonucleotide manufactured or obtained using a method of the present invention. Anti-sense oligonucleotides as described herein may be used in methods of therapy, for instance in treatment of individuals with the aim of preventing or curing (wholly or partially) a disorder associated with aberrant gene expression. Anti-sense oligonucleotides may be manufactured and/or used in preparation (i.e. manufacture or formulation) of a composition such as a medicament, pharmaceutical composition or drug. These may be administered to individuals. Thus, the present invention extends in various aspects not only to an oligonucleotide identified as having an anti-sense effect, in accordance with what is disclosed herein, but also a pharmaceutical composition, medicament, drug or other composition comprising such an oligonucleotide, a method comprising administration of such a composition to a patient (e.g. for down-regulating gene expression for instance in treatment, which may include preventative treatment, of a disorder associated with expression of mRNA), use of such a substance in manufacture of a composition for administration (e.g. for down-regulating expression of mRNA for instance in treatment of a disease associated with expression of an mRNA), and a method of making a pharmaceutical composition comprising admixing such a substance with a pharmaceutically acceptable excipient, vehicle or carrier, and optionally other ingredients.

Disorders associated with mRNA expression include disorders associated with aberrant gene expression, such as cancer, and disorders associated with expression of foreign genes such as infection with bacterial, viral or fungal pathogen. Any such disorder may be treated using anti-sense reagents as described herein.

Administration of an anti-sense oligonucleotide to an individual is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment (e.g. decisions on dosage, etc.) is within the responsibility of general practitioners and other medical doctors.

Pharmaceutical compositions according to the present invention, and for use in accordance with the present invention, may include, in addition to active ingredient, a pharmaceutically acceptable excipient, carrier, buffer, stabilizer or other materials well known to those skilled in the art. Such materials should be non-toxic and should not interfere with the efficacy of the active ingredient. The precise nature of the carrier or other material will depend on the route of administration, which may be oral, or by injection (e.g. cutaneous, subcutaneous or intravenous).

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, or Lactated Ringer's Injection. Preservatives, stabilizers, buffers, antioxidants and/or other additives may be included, as required.

Further aspects of the present invention provide methods of in situ hybridization and RNA structural analysis.

Aspects of the present invention will now be illustrated with reference to the accompanying figures described already above and experimental exemplification, by way of example and not limitation. Further aspects and embodiments will be apparent to those of ordinary skill in the art. All documents mentioned in this specification are hereby incorporated herein by reference.

### EXAMPLES

### MATERIALS AND METHODS

Reagents

Restriction Enzymes were from New England Biolabs, USA. Oligonucleotides were purchased from Interactiva, Germany. DYEnamic terminator sequencing kit was from Amersham Pharmacia Biotech, Sweden. pGEM-T vector and competent E. coli were from Promega, USA. Streptavidin coated paramagnetic beads (Dynabeads) were purchased from Dynal, Norway. DEPC was from Sigma, USA. PCR purification kit was from Qiagen, USA. Transfection reagents for expressing mRNA in vitro (LipofectAMINE 2000 and LipofectAMINE) were from Life Technologies, USA.

Rabbit β-globin cDNA was RT-PCR amplified directly from Rabbit globin mRNA purchased from Life Biotechnologies, Sweden. β-galactosidase cDNA was directly amplified from a LacZ plasmid. CGR95 full-length cDNA was cloned from rat brain HEK 293 cells (QBI-293A) were from Quantum Biotechnologies, USA.

Combinatorial Libraries

Four different combinatorial oligonucleotide libraries were constructed. For each library, oligonucleotides were synthesized separately and annealed in equal molar concentration (100 mM each) in 2×SSC (300 mM NaCl, 50 mM sodium citrate, pH 7) using a temperature touchdown program (94° C. for 3 min. and then 92° C. for 20 sec., 90° C. for 20 sec., 88° C. for 20 sec., and so on, till 30° C. for 20 sec.). The library was then stored at 4° C.

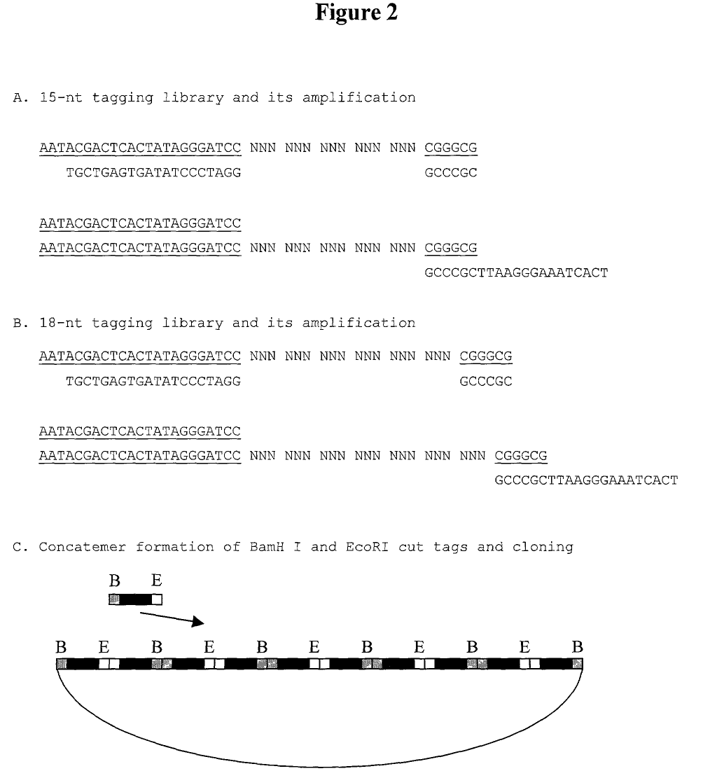

The first generation of the library contained 15 totally randomized nucleotides nested in two stretches of clumping sequences (FIG. 2A) whereas in the second generation library the randomized sequence was changed to 18 nt (FIG. 2B). Only the randomized portion of the plus strand was designed to be available for hybridization with mRNA samples at experimental temperatures (37° C. -40° C.).

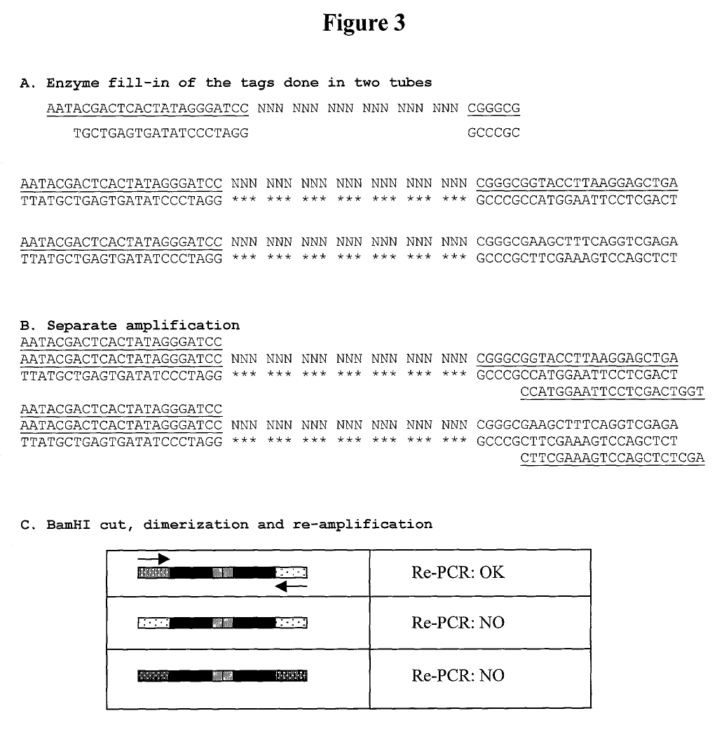

The clumping sequences were designed to facilitate the amplification of specific oligonucleotides after the selection procedures. The 3′ clumping sequences were made small so that they would afford minimal steric hindrance and give more flexibility to the random portion of the library. Amplification of the first and second generation of libraries can be done at single tag levels (FIG. 2C) or at di-tag levels (FIG. 3). The scheme for amplifying di-tags was tedious but it could be very helpful for handling short tags.

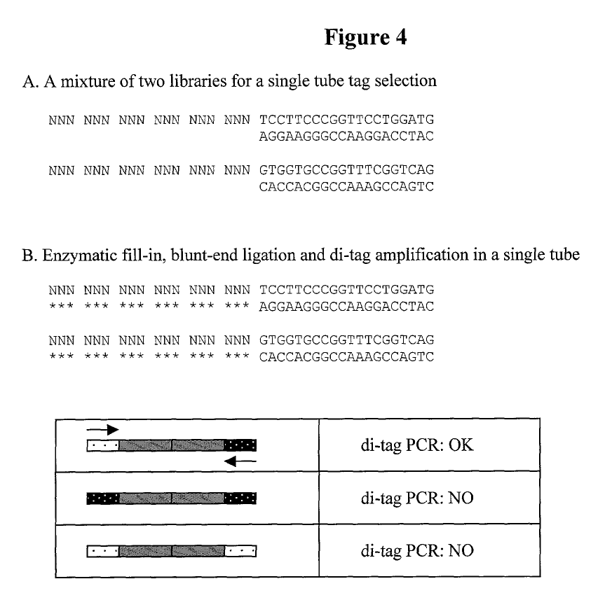

For high throughput analysis of the tags, concatemers of the tags were generated. Singly amplified tags were cleaved with EcoRI and BamHI. The tag fragments nested between the two sites were resolved on 20% polyacrylamide gel (acrylamide:bis-acrylamide =30:1). The DNA was cut out from the gel and eluted into 300 mM NaAc buffer (pH 5.2) overnight and precipitated together with 1 ml Glycoblue (15 mg/ml). The fragments were then resuspended into 20 ml H₂O and ligated with T4 ligase for 2 hours at RT. The ligation products were size-selected on 1% agarose gel to recover fragments that were in the range of 250 bp to 600 bp. Similar procedures can be used to form concatemers of the di-tags, but the enzyme used would be Kpn I and Hind III for libraries of first and second generation (FIG. 3) and Nla III or Msp I for the third generation library (FIG. 4).

A third generation of the library was constructed without the 5′ clumping site. In order to make the library amplifiable with PCR, a pair of such libraries was prepared so that they contain different 3′ clumping sequences. The library-pair can then be used as a single mixture for subsequent antisense oligonucleotide selection. The selected tags were then filled in by incubating 50 μl of reaction mix containing 1×PCR buffer, 100 μM dNTPs, 2.5 mM MgCl₂, 5 μl selected oligonucleotide tag, 2 μl fill-in primer and 0.5 units taq polymerase at 95° C. for 3 min., 37° C. for 3 min., 39° C. for 3 min., 42° C. for 3 min., 50° C. for 5 min., 7 min. The resulting blunt end was ligated in pairs (unwanted ligation blocked by 5′ modifications and 3′ phosphorylation). The di-tags were then amplified.

Preparation of Biotin Labelled mRNA

cDNA fragments were tagged with T7, T3, or Sp6 promoters during PCR amplification and were used to produce the corresponding mRNA (cRNA) by in vitro transcription reactions driven by T7, T3, or Sp6 RNA polymerases according to the procedures from the manufacturers, except for that all transcription reactions were supplemented with 0.1 mM biotin-UTP (Amersham Pharmacia Biotech, Sweden) in addition to 1 mM each of ATP, UTP, CTP, GTP. The products were normally analyzed using 1% agarose gel to control for the quality of mRNA.

RNA Immobilization and Oligonucleotide Selection

The MAST procedure is shown diagrammatically in FIG. 5. Typically, 100 ml of suspended Dynabeads was washed with 200 ml DEPC treated 2×SSC for 10 times. Then the beads were resuspended in 50 ml 5×SSC containing 5 mg biotin labelled mRNA and the binding reaction was allowed to proceed for 30 min. at RT with constant shaking. Afterwards the beads loaded with mRNA were washed 10 times in 5×SST (SSC solutions supplemented with 0.1% Tween-20). The beads were then resuspended in 100 ml 2×SST containing 1-2 ml of the combinatorial library and the hybridization of the immobilized mRNA and the oligonucleotides from the oligonucleotides library was allowed to proceed for 1 hr at 40° C. with constant shaking. The beads were sequentially washed with 1×SST for 10 times at 40° C. and 5 oligonucleotides times with 1×SSC at RT. The beads were then resuspended in 50 ml H₂O and boiled for 2 min. and the bound oligonucleotides were recovered in the aqueous phase. The recovered oligonucleotides were referred to as Accessible Site Tag (AST).

Amplification and Sequencing of Accessible Site Tags (AST)

One ml of recovered oligonucleotides was PCR amplified using the appropriate primers for each library. The PCR was carried out in the following thermocycles: one cycle of 94° C. for 2 min., 37° C. for 1 min., 40° C. for 1 min., 45° C. for 1 min., 50° C. for 35 cycles of 94° C. for 30 sec., 50° C. for 30 sec., and 72° C. for 30 sec. PCR prod tags) were purified with Qiagen PCR purification kit and subcloned into pGEM-T vectors. Sequencing was done by using the DYEnamic terminator sequencing kit according to the manufacturer's instructions. Comparison of the oligonucleotide sequences with the target gene was done using DNAStrider™ software.

Functional Analysis of Selected CGR95 Antisense Sequences

HEK293 cells were transfected with a plasmid expressing full length CGR95 mRNA. Antisense oligonucleotides (0.5 mM -5 mM) were used to treat the cells in the presence of LipofectAMINE 2000 according to manufacturer's protocol when the cells reach 85% -95% confluent. After the treatment total RNAs were prepared from the cells using SV total RNA purification kit from Promega and the level and integrity of the CGR95 mRNA was analyzed by Northern blot. Seven oligonucleotides selected according to the MAST data were tested. Scrambled or empirical testing selected oligonucleotides were used as controls. Oligonucleotides found to be effective were also tested by injection into rat brain in an in vivo locomotion assay.

### RESULTS AND DISCUSSION

Mapping of Accessibility of 122 Rabbit β-globin mRNA

The first 122 bp of the rabbit β-globin mRNA has been thoroughly interrogated for accessibility to antisense binding by an oligonucleotide array method in combination with RNase H assay and in vitro translation analysis (7) and other methods (10). β-globin mRNA was used as a model system to validate the current method of mRNA accessibility mapping.

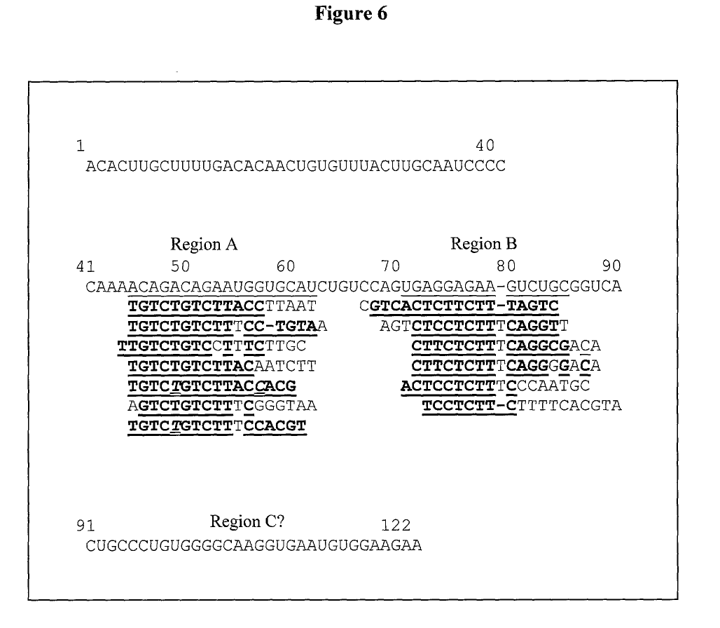

Two ml of the 100 mM combinatorial oligonucleotide library (1.2×10¹⁴ independent oligonucleotide molecules) was allowed to hybridize to 5 mg of bead-immobilized β-globin mRNA. As the random portion of the library was set to be 18-mer, the input amount corresponds to an abundance of 100 molecules/18mer. In order not to disrupt the authentic secondary structure of the mRNA, the hybridization conditions of this and all subsequent experiments were set to very mild conditions, i.e. 37° C. -40° C. in 2×SST. Although these conditions did not provide stringent enough selection for oligonucleotides that matches the target mRNA over their full length, the conditions were discriminative enough to eliminate most irrelevant oligonucleotides. After hybridization and washing, bound oligonucleotides, referred to as Accessible Site Tags (AST), were eluted into H2O at 100° C. for 2 min. and PCR amplified. After PCR amplification and cloning, 19 ASTs were sequenced and compared to β-globin mRNA (FIG. 6). Thirteen of the tags pinpoint to two regions of the 122 nt β-globin mRNA fragment (i.e. region A, nt 40-nt 62, and region B, nt 67- nt 85). The prediction of region nt 40-nt 62 is in very good coincidence with results from the scan array data (7), providing indication that this region is indeed an accessible region that can be detected by the present method as well as previous methods.

The identification of region nt 67-nt 85 also matched with the array-based results qualitatively, but differences emerged when the yields of AST (or hybridization yield in the array method) were compared. A similar number of ASTs was recovered for region B as compared with region A whereas in the array method region B gave substantially lower hybridization yield. Such a difference would result in that by the MAST method, region B will be assigned as a strongly accessible region where as by the array method, this region will be assigned as a marginally accessible region. Secondary structure modeling suggested that region B is a predominantly single-stranded region, with no significant intra-molecular double helix formation. This seems to be in better accordance with predictions done by the MAST method than the array method.

In array-based analysis (Milner et al. 1997), a third region (region C) of accessibility was observed and hybridization yield of this region seems to be even stronger then region B, but no comments or functional annotation was made for region C in Milner et al. (1997). Surprisingly, no ASTs representing this region have been recovered in our mapping. Indirect evidence indicates that the results from the present AST mapping are correct. An oligonucleotide (BG3) overlapping with region C has been tested in activity assays and showed no antisense activity (Milner et al. 1997). This agrees with the prediction from secondary structures that this region is sequestered in tight intra-molecular duplex and this duplex region covers the entire region C.

MAST Mapping a 1 kb β-galactosidase mRNA and 1.6 kb CGR95 mRNA

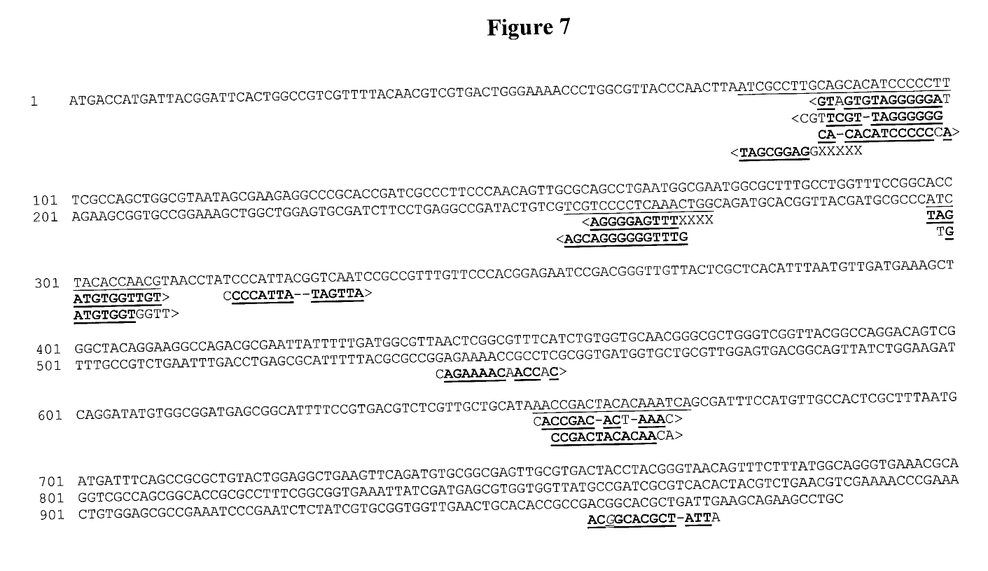

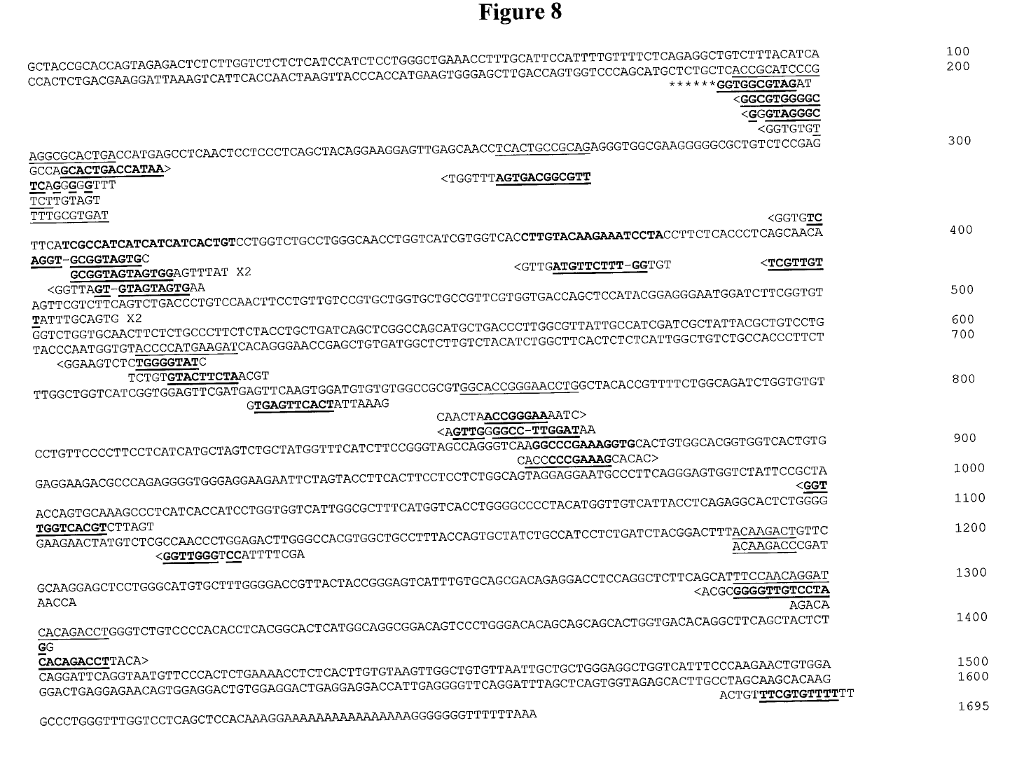

To further elucidate the applicability of the MAST method for mapping long mRNA, a 1 kb β-galactosidase mRNA and a 1.6 kb CGR95 mRNA were used as the model molecules. ASTs were isolated from the combinatorial oligonucleotide libraries and sequenced after PCR amplification. Four regions of the 1 kb β-galactosidase mRNA were marked with two or more independent AST (FIG. 7), and four regions were also mapped on CGR95 mRNA also (FIG. 8). Additional sites were covered with only one AST in both mRNA. The coverage of AST may not have reached saturation for these longer RNAs.

Functional Analysis of the MAST Mapped Regions

Seven olignucleotides were selected from the MAST mapped regions of CGR95, and tested for their antisense activity in cultured cells (Table 1). 0.5 μM oligonucleotides were used in the test. Seven antisense oligonucleotides, five selected from the five regions in CGR95 that were pinpointed by multiple ASTs and two from the region that were suggested by both an AST and emperical testing. All seven oligonucleotides were found to have significant antisense activity compared to only 10%-20% of empirically selected oligonucleotides. The MAST method appears to be much more powerful in selecting potent antisense reagents. Oligonucleotides selected by MAST were found to reduce the level of intact CGR95 mRNA by 50%-60% whereas the best oligonucleotide selected empirically only reduce the level of CGR95 mRNA by about 20%.

TABLE 1

| Name | Location |
|---|---|
| 1 | 195-211 |
| 2 | 255-271 |
| 3 | 306-322 |
| 4 | 614-630 |
| 5 | 757-773 |
| 6 | 998-1014 |
| 7 | 1291-1307 |
| A | 206-222 |
| B | 269-285 |
| Scrambled | — |

Table 1 shows CGR95 antisense oligonucleotides used in activity assays in HEK 293 cells. Oligonucleotide 1-7 were selected by MAST method. Control A sequence is complementary to a section spanning the putative translation initiation site. Control B was empirically selected oligonucleotide. A scrambled oligonucleotide with the same base content as oligonucleotide 1 was used as negative control.

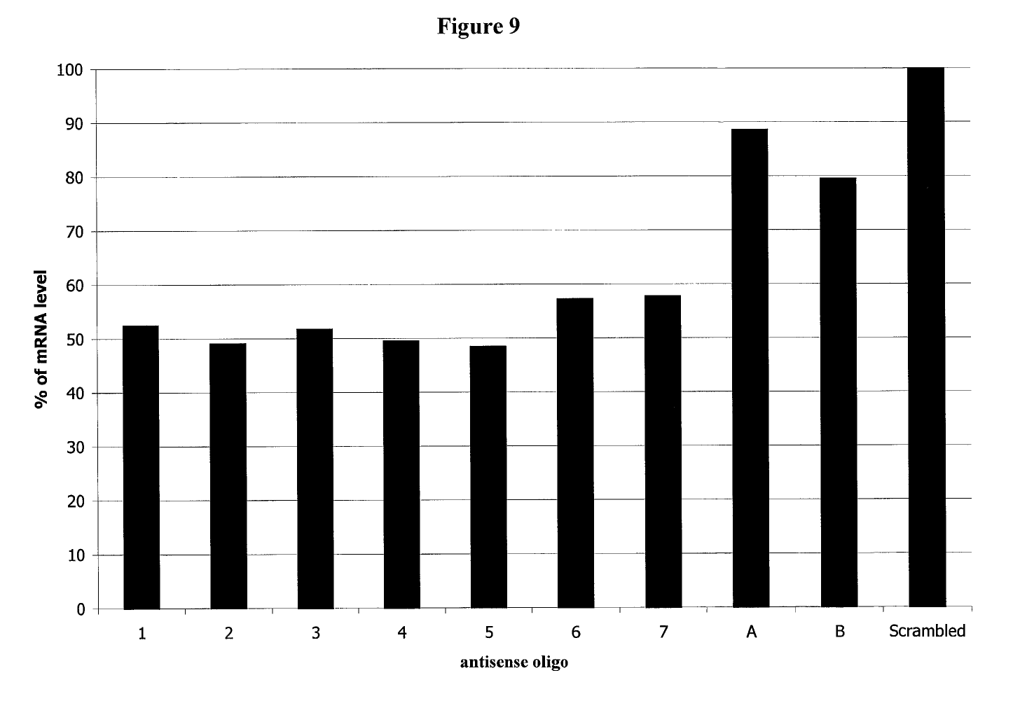

The antisense activity was also verified by in vivo experiments in rat brain (FIG. 9). Thus, an antisense oligonucleotide (number 1 in Table 1; 50.0 mg given twice a day intracerebroventricularly) targeted against the brain orphan G-protein coupled receptor, CGR95, was tested in a locomotor behaviour assay in rats. Rats were habituated in the open-field for 20 min before administration of the unselective dopamine receptor agonist apomorphine (1.6 mg/kg, s.c.), as indicated by the arrow in FIG. 9. Locomotor activity was recorded for additional 40 min. Data are shown as means±SEM based on 6 animals per group. A dependent two-way analysis of variance indicated that rats habituated during the habituation phase, but there was not a statistical significant difference between groups, suggesting no effects of antisense treatment per se on locomotor activity. After the injection of apomorphine the antisense-treated group displayed a decreased locomotor activity compared to controls, as shown by a statistical significant main effect of group (F(1, 10)=6.1, P=0.03). Control rats were injected with equal dose of a mismatched oligonucleotide or with vehicle only.

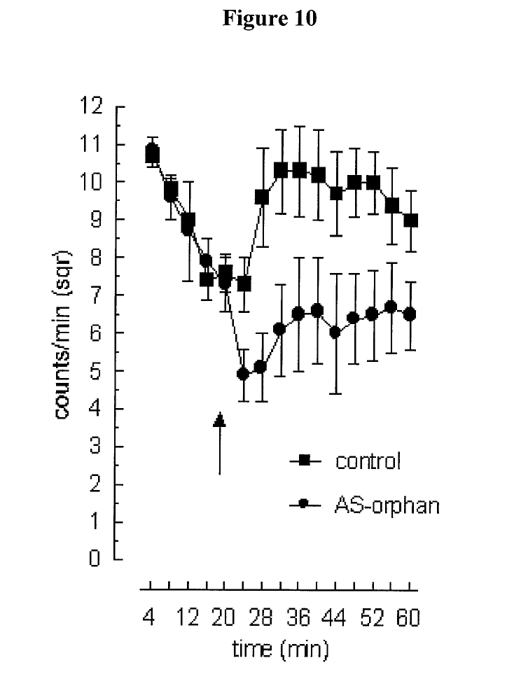

While antisense treated rats did not differ from control rats in baseline locomotor behaviour, the former responded differently to apomorphine (cf. FIG. 10). This indicates a functional interaction between CGR95 and apomorphine target(s).

### REFERENCES

1) Agrawal S & Zhao Q (1998) Curr Opin Chem Biol 2, 519-28

2) Wahlestedt C (1994) Trends Pharmacol Sci 15, 42-6

3) Matveeva O et al. (1998) Nat Biotechnol 16, 1374-5

4) Ho S P, et al. (1998) Nat Biotechnol 16, 59-63

5) Stull R A et al. (1996) Antisense Nucleic Acid Drug Dev 6, 221-8

6) Matveeva O et al. (1997) Nucleic Acids Res 25, 5010-6

7) Milner N et al. (1997) Nat Biotechnol 15, 537-41

8) Walton S P et al. (1999) Biotechnol Bioeng 65, 1-9

9) Patzel V & Sczakiel G (1998) Nat Biotechnol 16, 64-8

10) Patzel V et al. (1999) Nucleic Acids Res 27, 4328-34

What is claimed is:

1. A single-cycle method for identifying an accessible region of a native RNA, said method comprising, in sequence:
a) providing an in vitro reaction mixture comprising said RNA and a population of oligonucleotides, each oligonucleotide having a randomized portion, whereby said randomized portion can bind a complementary accessible region of said RNA if present;
b) selecting an oligonucleotide of said population that binds to said accessible region;
c) sequencing said randomized portion of said selected oligonucleotide; and
d) identifying the nucleotide sequence of said accessible region.

2. The method of claim 1, wherein all possible nucleotide sequences of said randomized portion are represented in said population of oligonucleotides.

3. The method of claim 1, wherein said RNA is mRNA.

4. The method of claim 1, wherein each oligonucleotide of said population comprises DNA.

5. The method of claim 4, wherein each oligonucleotide of said population comprises modified DNA.

6. The method of claim 1, wherein said randomized portion comprises 10 fully randomized nucleotides.

7. The method of claim 1, wherein said randomized portion consists of 13 to 18 fully randomized nucleotides.

8. The method of claim 1, wherein each oligonucleotide of said population further comprises a non-randomized portion adjacent said randomized portion.

9. The method of claim 8, wherein said randomized portion is at the 3′ end of said oligonucleotide and at least 4 contiguous non-randomized nucleotides are immediately 5′ of said randomized portion.

10. The method of claim 8, wherein said randomized portion is at the 5′ end of said oligonucleotide and at least 4 contiguous non-randomized nucleotides are immediately 3′ of said randomized portion.

11. The method of claim 8, wherein at least 4 contiguous non-randomized nucleotides are immediately 3′ of said randomized portion, and wherein at least 4 contiguous non-randomized nucleotides are immediately 5′ of said randomized portion.

12. The method of claim 8, wherein said reaction mixture comprises a blocking oligonucleotide that can hybridize to said non-randomized nucleotides.

13. The method of claim 1, wherein each oligonucleotide of said population has only one fully randomized portion.

14. The method of claim 1, wherein said population of oligonucleotides has not been previously selected to bind to said accessible region.

15. The method of claim 1, wherein said RNA is immobilized on a solid surface.

16. The method of claim 1, wherein said oligonucleotide is immobilized on a solid surface.

17. The method of claim 8, wherein said sequencing comprises amplifying said randomized portion to produce amplification products.

18. The method of claim 17, wherein said amplifying comprises annealing an amplification primer to said non-randomized nucleotides.

19. The method of claim 17, wherein said amplifying comprises hybridising a nucleic acid complementary to said selected oligonucleotide to produce a double-stranded nucleic acid for amplification.

20. The method of claim 17, wherein said amplifying comprises concatemerising said amplification products.

21. The method of claim 17, wherein said amplifying further comprises dimerizing said amplification products, and amplifying the resultant dimers.

22. The method of claim 1, wherein said identifying comprises analysing the sequences of at least 6 said randomized portions to identify said accessible region.

23. A single-cycle method for identifying accessible regions of at least two non-identical native RNA molecules, said method comprising, in sequence:
a) providing an in vitro reaction mixture comprising said RNA molecules and a population of oligonucleotides, each oligonucleotide having a randomized portion, whereby said randomized portion can bind complementary accessible regions on said RNA molecules if present;
b) selecting oligonucleotides of said population that bind to said accessible regions;
c) sequencing said randomized portions of said selected oligonucleotides; and
d) identifying the nucleotide sequence of said accessible regions.

24. A composition comprising a population of oligonucleotides, wherein each oligonucleotide of said population comprises a randomized portion and a non-randomized portion adjacent said randomized portion, and a plurality of blocking oligonucleotides hybridized to said non-randomized portion.

25. The composition of claim 24, further comprising a solid material onto which each oligonucleotide of said population is immobilized.

26. The composition of claim 24, further comprising native RNA, bound via an accessible region to a complementary randomized portion.

27. The composition of claim 26, further comprising a solid material onto which each oligonucleotide of said population is immobilized.

28. The composition of claim 26, further comprising a solid material onto which said native RNA is immobilized.

29. A kit comprising:
a) a first population of oligonucleotides, each oligonucleotide comprising a randomized portion and a non-randomized portion adjacent said randomized portion; and
b) a second population of blocking oligonucleotides complementary to said non-randomized portion.

30. A method for making an antisense oligonucleotide comprising identifying an accessible region of a native RNA by the method of claim 1, and synthesizing said antisense oligonucleotide, wherein said antisense oligonucleotide is complementary to said accessible region.

31. An antisense oligonucleotide obtained by the method of claim 30.

32. The antisense oligonucleotide of claim 31, wherein said antisense oligonucleotide is RNA.

33. The antisense oligonucleotide of claim 31, wherein said antisense oligonucleotide is DNA.

34. The antisense oligonucleotide of claim 31, wherein said antisense oligonucleotide is PNA.

35. A method for making a pharmaceutical composition comprising manufacturing an antisense oligonucleotide by the method of claim 30 and mixing said antisense oligonucleotide with a pharmaceutically suitable excipient.

36. A pharmaceutical composition obtained by the method of claim 35.

37. A method for treating a disorder associated with the expression of an mRNA, comprising administering the composition of claim 36 to a mammal.
